(19) United States
(12) Patent Application Publication
(10) Pub. No.: US 2006/0110828 A1
(43) Pub. Date: May 25, 2006

(54) Compositions and methods for selection of a pure population of cells from a mixed population

(75) Inventor: Uthayashanker R. Ezekiel, St. Louis, MO (US)

Correspondence Address: Joseph E. Zahner, 3646 Dover Place, St. Louis, MO, 63116, US

(73) Assignee: GeneProTech, Inc.

(21) Appl. No.: 10/997,203

(22) Filed: Nov. 24, 2004

(51) Int. Cl. C12N 5/08; C12N 15/87

(52) U.S. Cl. 435/455; 435/366; 435/368

(57) ABSTRACT

Disclosed are methods and compositions for deriving a pure population of differentiated cells from a stem cell. The method comprises transforming a stem cell with a gene construct containing a selectable marker under the control of a tissue- or cell-type-specific regulatory element, such that the gene construct integrates at a single gene locus, allowing the transformed stem cell to differentiate and give rise to a mixed population of differentiated and differentiating cells, applying a selection pressure to the mixed population, such that only the cell-type capable of driving the expression of the selectable marker can survive. Specifically disclosed is a method for selecting a pure population of neurons derived from embryonic stem cells by integrating at the HPRT site of the host stem cell genome a gene construct containing a polynucleotide encoding puromycin-N-acetyl-transferase operably linked to a necdin promoter, selecting for transformed integrants, differentiating the stem cells to form a mixture of cell-types, and selecting for neurons by applying puromycin to the mixture of cell types.

### GOVERNMENTAL SUPPORT

This work was supported by the U.S. Department of Health and Human Services/National Institutes of Health grant number R43 NS46133. The U.S. Government has certain rights in this invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

This invention relates generally to stem cell differentiation and cell therapy for regenerative medicine. This invention relates specifically to the purification of neurons from a population of cells derived from embryonic stem cells.

2. Summary of the Related Art

ES Cell Differentiation

Embryonic stem (“ES”) cells are capable of differentiating into myriad different lineages of cells, depending upon growth conditions and external factors added to the culture medium. Several model systems have been developed to drive the differentiation of ES cells into hematopoietic, neuronal, adipocytic and other cell types (se HQ Xian and DI Gottlieb, 2001, Trends Neurosci 24, 685-686; R M Schmitt, E Bruyns and HR Snodgrass, 1991, Genes Dev 5, 728-740; G Keller, M Kennedy, T Papayannopoulou and M V Wiles, 1993, Mol Cell Biol 13, 473-486, which are incorporated by reference). For research, diagnostic and therapeutic purposes, it is highly desirable to be able to generate a pure population of a single cell type from a mixture of cells of different lineages that are normally present after ES cell differentiation. For example, various methods for ES cell differentiation exist that result in the generation of hematopoietic and neuronal lineages of cells; however, no method exists that allows for the generation of either a pure hematopoietic or a pure neuronal cell type from the differentiating ES cells.

As a case in point, the addition of retinoic acid or fibroblast growth factor (FGF)-2 to ES cells results in the formation of neuronal-like cells, as defined by morphology, immunocytochemistry and transcript expression. These neouronal-like cells represent anywhere from 20% to 90% of the total cell population (see G Bain, D Kitchens, M Yao, J E Huettner and D I Gottlieb, 1995, Dev Biol 168, 342-357; A Fraichard, 0 Chassande, G Bilbaut, C Dehay, P Savatier and J Samarut, 1995, J Cell Sci 108, 3181-3188; S Okabe, K Forsberg-Nilsson, A C Spiro, M Segal and R D McKay, 1996, Mech Dev 59, 89-102; F Ciccolini and C N Svendsen, 1998, J Neurosci 18, 7869-7880, which are herein incorporated by reference).

Many scientific questions remain to be answered using stem cells for the treatment of diseases. One of the caveats of direct transplantation of stem cells, prior to any in vitro forced differentiation regimen, is the formation of ES cell-derived tumors (see M Li, L Pevny, R Lovell-Badge and A Smith, 1998, Curr Biol 8, 971-974; B Soria, E Roche, G Bema, T Leon-Quinto, J A Reig and F Martin, 2000, Diabetes 49, 157-162; S Liu, Y Qu, T J Stewart, M J Howard, S Chakrabortty, T F Holekamp and J W McDonald, 2000, Proc Natl Acad Sci 97, 6126-6131, which are herein incorporated by reference). Thus it is desirable to differentiate the stem cells into differentiated derivatives (preferably terminally mature differentiated cells) before transplanting them into a recipient host. Results from a number of animal studies have shown that differentiated stem cells, after implantation in adult animals, do not cause significant tumor formation. Thus, it is highly desirable to have a relatively pure population of differentiated cell types in vitro, for subsequent use in research, diagnostics and therapy.

### Cell Selection and Gene Integration

It is generally known in the cell biological arts that gene promoters whose expression is limited to specific cell types may be useful to drive expression of a selection marker to allow for the survival of only a specific cell type in which the promoter is active and allows for the elimination of all other cell types when an appropriate selection pressure is applied. These methods generally relay upon the random integration of a gene construct into the genome of the recipient host cell. The gene construct generally contains a cell-type specific promoter fused to a gene that confers resistance to a toxin.

Random integration of an ectopic gene construct can cause a myriad of technical genetic problems due to positional effects and interference with essential genes. Use of site-specific integration is more desirable since many problems related to random integration, such as inactivation of genes, can be avoided. A preferred single site locus for integration is the hypoxanthine phosphoribosyl transferase (“HPRT”) locus. The HPRT gene is expressed by ES cells and during all stages of development, thereby suggesting that the locus remains in an open chromatin configuration and constitutively active (S K Bronson, E G Plaehn, K D Kluckman, J R Hagaman, N Maeda and O Smithies, 1996, Proc Natl Acad Sci 93, 9067-9072). Thus, HPRT-specific promoters and enhancers are thought to retain their natural specificity in differentiated tissues and cell types (Smithies et al. 1996 Proc Natl Acad Sci USA 93, 9067-9072; JR Shaw-White, N Denko, L Albers, T C Doetschman and J R Stringer, 1993, Transgenic Res 2, 1-13.

The protein encoded by the HPRT gene is involved in the salvage pathway of nucleotide metabolism. This gene has nine exons that are spread over a 33 Kb region of the X chromosome (D W Melton, D S Konecki, J Brennand and C T Caskey, 1984, Proc Natl Acad Sci 81, 2147-2151). A cell with a mutated HPRT gene will survive when grown in the presence of the nucleotide analogue, 6-thioguanine (6-TG), but cells with a wild type HPRT gene will not (J R Shaw-White, N Denko, L Albers, T C Doetschman and J R Stringer, 1993, Transgenic Res 2, 1-13). The presence of 6-TG in the culture media causes incorporation of 6-TG into the nucleotide pool which, when incorporated by the HPRT enzyme, leads to cell death. When the HPRT gene is disrupted, the cells survive in 6-TG because the nucleotide analogue will not be incorporated into the DNA. Therefore, 6-TG selection offers a powerful way to screen cells for insertions of selected genes. INSERT Methods used in selection of specific cell lineages from differentiated embryonic stem cells

### Selection of Specific Cell Lineages from Differentiated Embryonic Stem Cells

Several studies have been reported of stem cell differentiation and differentiated cell isolation using genetic markers. Pages and coworkers (J Cell Science 115:2075, 2002) have described stable embryonic stem cell transfection using a construct with an endothelial promoter linked to puromycin selection and enhanced green fluorescent protein (EGFP) reporter genes that, upon differentiation and selection, express EGFP in cells of endothelial origin. Gold and Lebkowski (U.S. Pat. No. 6,576,464) teach an effector gene regulated by a transcriptional control element that causes gene expression in undifferentiated cells of a population whereby effector gene (e.g., toxin) expression results in depletion of the undifferentiated cells. To enrich for cardiomyocytes from differentiated embryonic stem cells, Klug et al (J Clin Invest 98:216, 1996) engineered ES cells to contain a stable fusion gene of the cardiac myosin heavy chain promoter driving the aminoglycoside phosphotransferase gene (which thereby confers resistance to the drug G418) and these cells were differentiated and positively selected in vitro using G418 with results being survival of cardiomyoctyes only. Levesque (U.S. Pat. No. 6,087,168) showed transdifferentiation of epidermal cells into neuronal cells using a two-fold process: 1) expression of a neuronal transcriptional gene in the epidermal cell; and 2) suppression of negative regulators of neuronal differentiation in the epidermal cells by addition of antisense oligonucleotides. Stein et al (Proc Natl Acad Sci 96:7294, 1999) reported osteoblast-targeted gene expression controlled by a bone-specific osteocalcin promoter after systemic transplantation of heterogeneous mouse marrow cells. Brustle et al (FASEB J. 2004 Oct. 14:Epub ahead of print) isolated murine ES cell clones stably transfected with a construct encoding the beta-galactosidase-neomycin-phosphotransferase fusion protein under control of the 2′3′-cyclic nucleotide 3′-phosphodiesterase (CNP) promoter that were differentiated into bipotential glial precursors and subsequently induced at the CNP-positive stage and selected with neomycin to a homogenous population of a pre-oligodendrocytic phenotype.

### SUMMARY OF THE INVENTION

Heretofore, the skilled artisan has relied solely upon random multi-copy integration to select for differentiated cell types. Such an approach presents many problems, including gene inactivation and inappropriate gene expression. Thus, such an approach is not tenable for use in producing pure and well-characterized differentiated cells for regenerative medicine research and therapy. The inventor has discovered that targeted insertion of a single copy of a selectable gene cassette can work in a selection schema for specific types of differentiated cells.

In one embodiment, the invention is directed to a method for selecting a particular desired type of eukaryotic cell from a mixture of cell-types. Preferably the mixture of cell types is derived from a progenitor cell, such as an embryonic stem cell, that has undergone or is in the process of differentiation. The mixture of cell types is contacted with an agent that selectively inhibits the proliferation of or kills those cell types in the mixture that are not desired, while allowing the particular desired type of eukarytotic cell to survive. This is negative selective pressure. Alternatively, the mixture of cell types is contacted with an agent that selectively promotes the proliferation or survival of the particular desired type of eukaryotic cell. This is positive selective pressure.

In a preferred aspect of this embodiment, the progenitor cell contains a gene construct comprising a polynucleotide, which encodes a polypeptide that inactivates the agent used as the selective pressure, the polynucleotide operably linked to a regulatory element. A preferred regulatory element is a tissue-specific gene promoter, such as for example, a neuron-specific promoter, a beta-islet cell-specific promoter, a muscle-specific promoter, a cardiomyocyte-specific promoter, a bone homeostasis-specific promoter, a leukocyte-specific promoter, a vascular endothelial cell-specific promoter, a hepatocyte-specific promoter and a lung epithelial cell-specific promoter. Non-limiting examples of useful promoters include necdin promoter, cardiac actin promoter, albumin promoter, and insulin promoter. The skilled artisan, in the practice of this invention, is capable of readily recognizing that myriad other gene promoters may be used, depending on the nature of the particular desired cell type.

Examples of agents that are useful to produce a selective pressure in the practice of this invention include, but are not limited to, puromycin, hygromycin, neomycin, zeocine, tetracycline and hypoxanthine. Preferably, the polynucleotide encodes a polypeptide appropriately matched to the agent. For example, when puromycin is used as the agent, the polynucleotide may encode the polypeptide puromycin-N-acetyl-transferase.

In a more preferred aspect of this embodiment, the gene construct is integrated into a single locus of the progenitor cell genome, such as the hypoxanthine phosphoribosyl transferase (“HPRT”) locus. Additionally, several rounds of selective pressure may be applied to the progenitor cell and the consequential differentiated cell types derived from that progenitor cell. A first selective pressure can be applied to the progenitor cell such that only a progenitor cell having the gene construct integrated into a predetermined single site (e.g., HPRT site) to proliferate to produce a clonal population. Then the cells within the clonal population are allowed to differentiate into one or more specific cell types to produce the mixture of cell-types; followed by applying a second selective pressure (agent) to the mixture of cell-types, such that the particular desired cell-type survives and the undesired cell types do not survive.

In another embodiment, the invention is directed to a gene construct comprising (1) a tissue-specific promoter, (2) a polynucleotide which encodes a polypeptide capable of inactivating an agent, and (3) a nucleotide sequence that is homologous to a region of a host genome. Preferably, the agent is any one or more of puromycin, hygromycin, neomycin, zeocine, tetracycline and hypoxanthine; the tissue-specific promoter is any one or more of a neuron-specific promoter, a beta-islet cell-specific promoter, a muscle-specific promoter, a cardiomyocyte-specific promoter, a bone homeostasis-specific promoter, a leukocyte-specific promoter, a vascular endothelial cell-specific promoter, a hepatocyte-specific promoter and a lung epithelial cell-specific promoter; and the region of a host genome is a single site wherein a gene that is expressed in the progenitor cell as well as the particular desired type of eukaryotic cell. More preferably, the promoter is a necdin promoter (e.g. SEQ ID NO:3), the polypeptide is puromycin-N-acetyl-transferase, which metabolizes puromycin, and the region of the host genome is the HPRT locus.

In another embodiment, the invention is drawn to a progenitor cell comprising the gene construct (supra). The preferred progenitor cell is an embryonic stem cell.

In yet another embodiment, the invention is drawn to a particular desired eukaryotic cell type comprising the gene construct (supra). The preferred particular desired eukaryotic cell type is a neuron.

### BRIEF DESCRIPTION OF THE DRAWINGS

FIG. 1 depicts a schematic representation of the negative selection of specific cell types from a mixture of cell types.

FIG. 2 depicts maps of the HPRT gene locus (A), the pUT1 plasmid (B) and the pHPRT targeting vector for integration of the selectable construct into the single HPRT locus of the genome (C).

FIG. 3 depicts the identification of HPRT integrants in a genome; wild-type HPRT locus (A), HPRT locus containing the selectable construct (B), and a southern blot panel depicting the wild-type genome (lane 1, C) and transformed genome (lane 2, C).

FIG. 4 depicts the quantification of beta galactosidase activity in undifferentiated ES cells and in ES cells that have differentiated at various times post differentiation.

FIG. 5 depicts neurons derived from ES cells at day 12 of differentiation. Panel A shows neuron-specific MAP2 (a+b) staining; panel B shows bright field image of same field as shown in A; panel C shows DAPI-stained nuclei of same field as shown in A and B.

FIG. 6 shows neuron cells derived from ES cells containing a necdin-driven Lac Z construct; panel A shows beta-galactosidase staining and panel B shows DAPI-stained nuclei of the same filed of cells as in panel A.

FIG. 7 depicts the construction of the HPN targeting vector. Panel A depicts pUT2 vector, panel B depicts the PILPN vector containing the necdin promoter linked to a puromycin slection marker, IRES and LacZ gene, panel C depictis the HPN vector that contains the PILPN vector within pHPRT for targeting to the HPRT locus of the genome.

### DETAILED DESCRIPTION OF VARIOUS EMBODIMENTS

The following examples demonstrate particular preferred embodiments of the invention and are meant to be illustrative and not limiting in any way. The skilled artisan in the practice of this invention will readily recognize other equivalents and specific ways to accomplish the central tenets of this invention. The metes and bounds of the invention are not defined by this specification, but by the claims, which follow the examples.

The invention is directed to compositions and methods of selecting for a specific cell type from a mixture of cell types. The method involves transforming a progenitor cell, as defined herein, in a re-selected single site in the genome with a gene construct that allows for the selection of a desired cell type from a population of multiple different cells types derived from the progenitor cell upon the application of an external selective pressure. The compositions include, but are not limited to, the gene construct, the progenitor cell containing the gene construct, and the desired cell type containing the gene construct.

### Definifions

As used herein, the term “gene construct” means an isolated recombinant polynucleotide comprising a cell-type-specific or tissue-specific regulatory element (promoter, enhancer, silencer, and the like), a polynucleotide encoding a polypeptide that confers resistance to an agent, and regions of polynucleotide homology to a specific site within a host cell genome, to allow for integration of the gene construct into the specific site by homologous recombination.

As used herein, the term “progenitor” or “progenitor cell” shall have a broader meaning than that which is generally known in the art to mean any and all type of cell capable of further differentiation to give rise to a more terminally differentiated cell type, either immediately or several developmental steps downstream. The progenitor may be an embryonic stem (“ES”) cell, an adult stem cell, an art recognized progenitor cell, a totipotent cell, a pluripotent cell, and a multipotent cell.

The term “visual read-out” means any means of visually identifying a cell, such as for example the development of a color (e.g., X-gal production from beta-galactosidase), a burst of light (e.g., ATP-luciferase), and the like.

As used herein, the term “promoter” shall have a broader meaning than that which is generally known in the art to mean any and all type of genetic cis-regulatory element capable of regulating expression of a proximal gene or other polynucleotide. The promoter may be an enhancer element, a silencer element, an art recognized promoter, and the like.

### EXAMPLE I

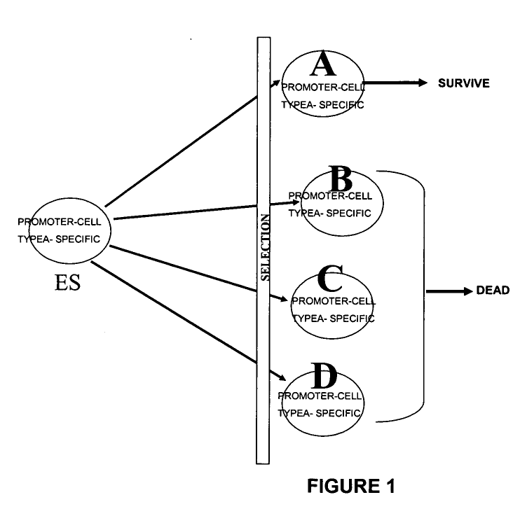

Using promoters whose expression is limited to specific cell types and that drive expression of specific selection markers allow for the survival of only the specific cell type in which the promoter is active and causes elimination of all other cell types when appropriate selection pressure is applied (FIG. 1). A specific locus in the ES cell is targeted with a selection marker and a reporter gene under the control of the desired cell type promoter. After differentiation, the appropriate selection condition is applied to the cell culture thereby allowing the survival of the specific cell type of interest in which the promoter is exclusively expressed. Expression of a reporter gene aids in the quantitation and tracking of the specific cell type and in determination of efficiency of the selection pressure. The selection pressure is maintained and then removed when the specific goal is achieved. This genetically modified ES cell clone is maintained at an undifferentiated stage by growth under appropriate media and conditions. Whenever needed, the cells are differentiated and selection pressure applied for enrichment of specific cell types. To show the applicability of this technology and to demonstrate that a specific cell type is indeed selectable from a mixture of cells, neuronal differentiation of ES cells mediated by retinoic acid is used as a model system. Differentiation of ES cells by retinoic acid leads to the formation of neuronal precursor cells followed by development of neurons, glial cells and astrocytes (G Bain, D Kitchens, M Yao, J E Huettner and D I Gottlieb, 1995, Dev Biol 168, 342-357; A Fraichard, O Chassande, G Bilbaut, C Dehay, P Savatier and J Samarut, 1995, J Cell Sci 108, 3181-3188). With an appropriate promoter, selection and reporter system, different types of ES cell lines may be generated which is a valuable system for differentiation-related studies and for selection of pure cell types.

### HPRT Targeting Vector Construction

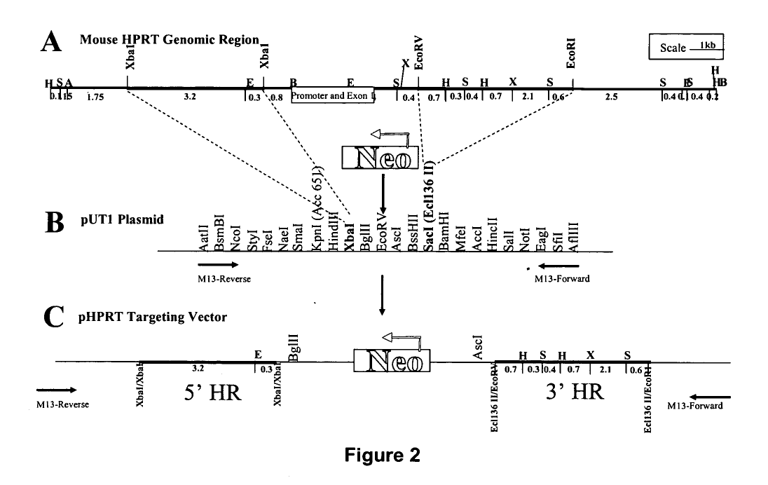

Two modified pUC 19 plasmids were engineered and used as the backbone for target vector construction. Plasmid pUC19 was digested by AatII and AflII restriction enzymes resulting in the production of two DNA fragments: 1) a 0.875 Kb fragment which contains the polylinker cloning site and the lacZ gene of pUC19; and 2) a 1.81 Kb fragment which contains the β-lactamase gene and the origin of replication. To the 1.81 Kb DNA fragment, several restriction sites were added by sequential ligation of oligolinkers thereby creating plasmid pUT1 (FIG. 2B).

FIG. 2A is a map of the mouse HPRT genomic locus. The promoter and exon 1 of the HPRT gene is in the central region and designated by the green box (FIG. 2A). The pUT1 backbone (FIG. 2B) was used to construct the targeting vector. To make the targeting vector (FIG. 2C), the 5′ and 3′ homologous regions flanking the 5′ region of the promoter (red line) and the 3′ region of exon 1 (blue line) were used (FIGS. 2A and 2C). Transcription of the HPRT gene requires the promoter region (green box). The targeting vector was designed such that homologous recombination of this vector at the HPRT locus eliminates the promoter-exon 1 region (green box) thereby completely eliminating HPRT gene expression.

The strategy utilized for construction of the HPRT targeting vector follows. Plasmid pUT1 (FIG. 2B) was used as a backbone to engineer the targeting vector (FIG. 2C). The source of the HPRT gene was a BAC clone identified by screening a BAC library made from 129/SvJ mouse genomic DNA. The BAC DNA was subcloned and relevant genomic regions were used to construct the HPRT targeting vector (FIG. 2C). The targeting vector was constructed from the 3.5 Kb XbaI DNA fragment located in the 5′ upstream region of the promoter (red lines in FIGS. 2A and 2C) and from a 4.8 Kb EcoRV and EcoRI DNA fragment located in the 3′ region downstream of exon 1 (blue lines in FIGS. 2A and 2C). The 5′ XbaI fragment was cloned into a pUT1 plasmid at the Xba1 site and 3′ DNA fragment EcoRV-EcoRI subjected to a fill-in reaction and cloned at the Ecl136II site that created a blunt end. At the EcoRV site of pUT1 containing 5′ and 3′ arms (FIG. 2B), a Neo^{r} selection gene was cloned. The presence of the phosphoglycerate kinase (Pgk) promoter allows for constitutive expression of the Neo^{r} gene. Addition of the antibiotic G418 to cells containing a Neo^{r} marker blocks protein synthesis by inhibition of ribosomal function. Cells expressing the Neo^{r} marker survive when grown in the presence of G418 because the Neo^{r} gene causes detoxification of G418 thereby allowing for cell growth.

### Transfection of ES Cells with the HPRT Targeting Vector

ES cells were electroporated (180 V, 500 uF) with 10 μg linearized HPRT targeting vector (FIG. 2C). After electroporation, ES cells were cultured on a fibroblast feeder layer at 37° C. and 7.5% CO₂ in standard ES cell culture media of Dulbecco's minimal essential medium with high glucose, L-glutamine (DMEM: Invitrogen, Carlsbad, Calif.) plus 1 mM sodium pyruvate, 10% fetal calf serum, 100 μM beta-mercaptoethanol and 1000 U/ml leukocyte inhibitory factor (ESGRO®, Chemicon, Temecula, Calif.). After 24 h, G418 selection was performed by G418 (400 μg/ml) addition and the cells were allowed to grow for 5 days. Only cells transfected with the HPRT targeting vector survived in the presence of G418. After 5 days, 30 μM 6-TG was added to the standard ES cell culture media and cells were grown for 7-10 days. Only cells with a mutated HPRT gene grew in 6-TG.

### Selection of Cells Containing the Targeted HPRT Gene

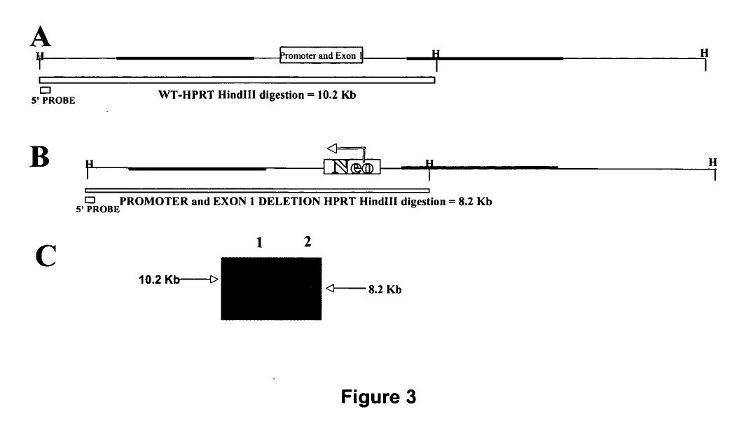

To identify that the mutation in the HPRT gene was due to gene targeting, a Southern blot was performed to verify that a homologous recombination event occurred. After 8-10 days, surviving colonies were picked and grown in 0.1% gelatin-coated microwells without 6-TG but in the presence of G418. When the cells reached confluence, genomic DNA was isolated using a standard protocol (Mouse Genetics and Transgenics, Oxford University Press, Oxford 2002; Cell Biology. A Laboratory Handbook, Academic Press, Boston 2002). A 5′ probe was made by PCR amplification of the 200 bp DNA fragment from the region between HindIII and Acc651. This probe is depicted as a black box on the 5′ end of FIGS. 3A, 3B and 3D. FIG. 3 shows the screening strategy for the targeting event. The 5′ probe was labeled with deoxycytidine 5′-[α-³²P] triphosphate and Rediprime® II DNA labeling system (Amersham, Biotech, Piscataway, N.J.). Genomic DNA was digested with HindIII and electrophoresed on an agarose gel and blotted onto a nylon membrane followed by hybridization with the labeled 5′ probe (FIG. 3C). As depicted by a turquoise box in the HPRT map (FIG. 2A), HindIII digestion of wild type genomic DNA produced a fragment of 10.2 Kb size by Southern blot using the 5′ labeled probe (FIG. 3C, lane 1). The targeted HPRT region has a deletion of the promoter-exon 1 region (4 Kb) and a replacement of a Neo^{r} selection marker cassette (˜2 Kb size). Therefore, HindIII digestion of the targeted HPRT gene resulted in an 8.2 Kb DNA fragment (FIG. 3C, lane 2). A Southern blot using a probe specific for the neomycin region resulted in the same size band (8.2 Kb) in genomic DNA from targeted cells. This is essential because a random integration event shows more than a single band. From a total of 15 colonies obtained, 12 had the correct targeting result based upon the use of the 5′ and neomycin probes and Southern analysis. These results show that 6-TG selection is a powerful method for the identification of HPRT targeting events.

### Characterization of the Necdin Promoter in ES Cells

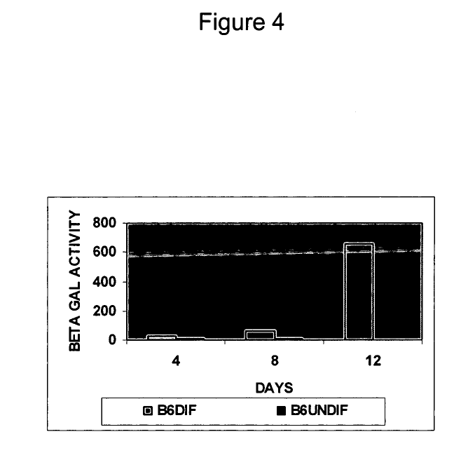

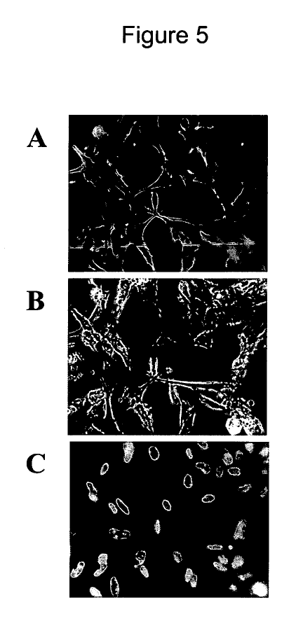

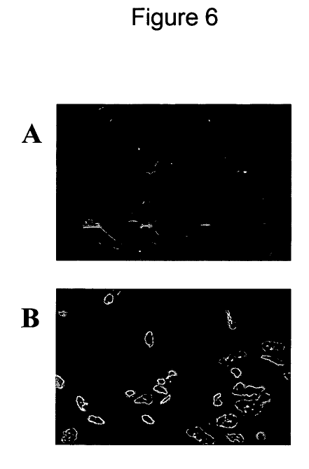

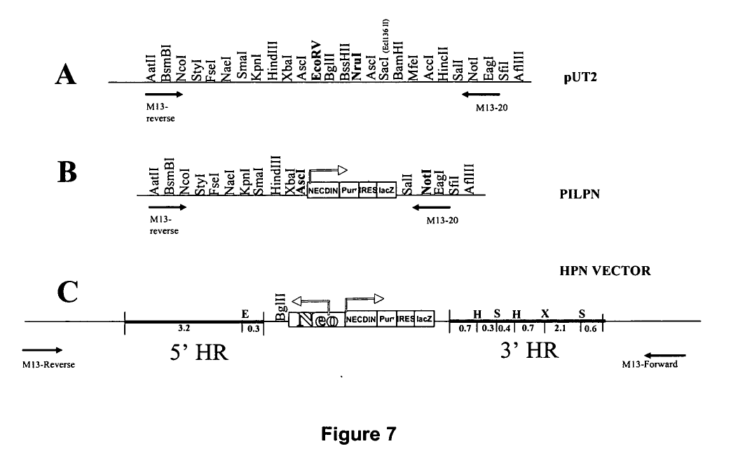

The promoter used in this example is necdin. According to Uetsuki et al (J Biol Chem 271, 918-924, 1996.) a 844 bp DNA fragment upstream of the transcription initiation start site for the necdin gene contains promoter activity in differentiated neuronal cells. The necdin gene is expressed in all post-mitotic neurons (K. Maruyama, M. Usami, T. Aizawa and K. Yoshikawa. 1991. Biochem Biophys Res Commun 178, 291-296.). For the present study, the necdin promoter region was cloned from a mouse genomic DNA by PCR amplification of a 951 bp of DNA fragment upstream of the translation initiation site. The primers for necdin amplification were chosen from the mouse necdin sequence available from GenBank (accession number D76440). The forward primer corresponds to nucleotide numbers 1-26 (SEQ ID NO:1-5′GATCATTTTCCACTAGAATCTTAACG3′) and the reverse primer corresponds to nucleotide numbers 956-937 (SEQ ID NO:2-5′TCTGATCCGAAGGCGCAGAC3′). A PCR reaction was done using Pwo DNA polymerase (Roche Applied Science, Indianapolis, Ind.) The PCR amplification reaction was performed according to manufacturer's guidelines. The amplified DNA fragment was cloned into the NruI/EcoRV site of the pUT2 vector (FIG. 7A). The cloned necdin promoter region was verified by sequencing of the region and by restriction enzyme digestion. The plasmid containing the necdin promoter was named pNecdin. A 3.6 kb lacZ-containing DNA fragment from pCMV beta (Clontech,) and was cloned at the 3′ end of necdin, to this plasmid a DNA fragment containing drug selction marker Neo fragment which was under the control of pgk promoter was also cloned, creating pNecdinlacZ. To confirm that necdin is specifically expressed in neurons, ES cells were transfected with pNecdinlacZ which drives the beta-galactosidase gene. A stable ES cell line was selected (ES cell line B6). This ES cell line was allowed to differentiate using the 4−/4+ retinoic acid protocol (Gottlieb et al. 1995), After 4+ RA treatment, the ES cells were plated on gelatin-coated tissue culture plastic plates and the expression of beta-galactosidase was analyzed by immunocytochemistry and also by a chemiluminescence assay (FIGS. 4, 6). FIG. 5 shows Day 12 differentiated ES cells that reacted with MAP2(a+b) antibody which is a marker for neurons. The cells with neuronal morphology were also reactive with beta-galactosidase antibody (FIG. 6) indicating that the necdin promoter drives reporter gene expression in neurons. Cells was lysed (100 mM potassium phosphate, pH 7.8, 0.2% (v/v) Triton X-100, 1 mM dithiothreitol) and lysates obtained from different time points (days of culture for ES cell differentiation). Beta-galactosidase activity was measured using beta-galacton, a chemiluminogenic substrate (I Bronstein, B Edwards and JC Voyta, 1989, J Biolumin Chemilumin 4, 99-111) that was a very sensitive detection method for beta-galactosidase activity. Activity was normalized to the amount of protein in the lysate using the bicinchroonic method (Pierce Chemicals, Rockford, Ill.). The quantitation by chemiluminescence for beta-glactosidase expression showed that after differentiation the activity of beta-galactosidase increased from day 4 to day 12 (FIG. 4) which correlated with the appearance of neuron-like cells. Of importance, no expression of beta-galactosidase activity was detected by chemiluminescence in undifferentiated B6 ES cells (FIG. 4).

### Stable ES Cells Expressing the Puromycin:Beta-Galactosidase Cassette under Necdin Promoter Control

The targeting vector PILPN consisting of a necdin promoter driving a puromycin selection marker and beta-galactosidase expression marker was constructed (FIG. 7), Vector construction utilized cloning of the internal ribosomal entry sequence (IRES) DNA fragment with the multiple cloning site (MCS) from pIRES (Clontech, Palo Alto) into a pUT2 plasmid vector (FIG. 7A). The pIRES plasmid was digested with PstI followed by a T4 DNA polymerase reaction to create blunt ends. This DNA molecule was digested with SalI and gel purified. The resultant 899 bp nucleotide fragment contained the IRES, and this fragment was cloned in to pUT2 vector at the NruI and SalI restriction sites and this plasmid was called PGIRES. Next, SmaI and SalI digestion of the pCMV beta vector produced a 3.6 kb β-galactosidase containing DNA fragment which was cloned into a BamH 1 site blunt ended by a T4 DNA polymerase reaction at a SalI site. The resulting plasmid was called PIL. In the third step, the DNA fragment containing the puromycin resistance gene (puromycin-N-acetyl-transferase gene [Pur^{r}] from the pPUR vector [Clontech, Palo Alto, Calif.]) was cloned into the PIL vector. The DNA fragment containing Pur^{r}-was isolated by digestion with AvrII and MfeI restriction enzymes. The isolated 919 bp DNA fragment was cloned into PGRES at the NheI and EcoRI I site. The resulting plasmid was called PILP. In the fourth step, the necdin promoter was isolated from the pNecdin vector by digestion with AscI and Ecl136 II and the resulting 925 bp DNA fragment was cloned into the AscI-EcoRV site of PILP. The plasmid was called PILPN (FIG. 7B). To make stable cell lines, the PILPN vector was linearized by SfiI digestion. The linearized PILP targeting vector was co-eletroporated with a plasmid containing a neomycin selection marker cassette that was linearized by NotI restriction digestion. After electroporation, ES cells were cultured on a fibroblast feeder layer at 37° C. and 7.5% CO₂ in standard ES cell culture media containing leukocyte inhibitory factor. Six stable lines were selected by neomycin selection. After 24 hours, G418 selection was performed by addition of ES cell media containing G418 (400 μg/ml) and cells were allowed to grow for 5 days. Only cells transfected with plasmid survived in the presence of G418. After 8-10 days, surviving colonies were picked and grown on 0.1% gelatin-coated plates. The presence of integrated PILP targeting vector in the ES cell lines was verified by polymerase chain reaction (PCR) analysis. PCR was performed using primer pairs specific for puromycin and lacZ genes. Six clones were identified as positive PILP targeted clones. ES cell line-6 has been further used for experimental strategies.

### Differentiation of ES Cell Line 6 Usage

ES cell line 6 was allowed to differentiate by 4−/4+ embryoid body formation and retinoic acid treatment (G Bain and DI Gottlieb, 1998, Perspect Dev Neurobiol 5, 175-178). At day 7 of differentiation, one set of differentiating cells received puromycin. At day 10, the selection pressure was stopped and the cells that received selection and those that did not (control) were analyzed for enrichment of neuronal cells. The results indicated that puromycin allowed only for neuronal cell survival and eliminated other cell types. This example shows that by use of specific promoters, single cell types can be enriched from a mixed population. The appropriate selection condition was applied to the cell culture thereby allowing the survival of the specific cell type of interest in which the promoter was exclusively expressed. The experiment described here allowed for the enrichment of a particular cell type, that is neuron. Expression of a reporter gene aided in the quantitation and tracking of the specific cell type and in determination of efficiency of the selection pressure. Comparison of beta-galactosidase activity was performed with or without selection for ES cell line-6 at different days after initiation of differentiation (days 10,12 and 16). The cells that had undergone selection had several fold increase in beta-galactosidase activity compared to cells that did not undergo selection. Cells that had undergone selection were specifically neuronal cells as identified by immunocytochemistry for MAP2 with MAP2 antibody and were not astrocytes or oligodendrocytes as determined by GFAP and O4 antibody immunocytochemistry. Cells with no selection pressure were identified as neurons, astrocytes and oligodendrocytes by immunocytochemistry. This invention shows that a particular lineage can be genetically selected.

### EXAMPLE II

Differentiation and Selection of Neurons from a Stable ES Cell Line Expressing Puromycin Selection and Beta-Galactosidase Expression Cassette under Necdin Promoter Inserted as a Single Copy at the HPRT Locus is described.

The vector PILPN is digested with AscI and SfiI and blunt ended by T4 DNA polymerase. The resultant DNA fragment is cloned at the blunt ended AscI site in the pHPRT vector. The resulting targeting vector is called HPN FIG. 7C). The vector HPN is linearized with SfiI and electroporated into ES cells. For a single copy insertion at the HPRT locus, the ES cells are first selected on G418 after 24 hours of electroporation. The surviving colonies formed by this selection indicate stable cells formed due to the integration of the targeted vector. The integration could be random or a specific homologous recombination at HPRT locus. To select for HPRT targeted clone, the colonies were selected on 6-thioguanine. Only the HPRT targeted clones survive the selection. The surviving colonies were expanded and the DNA is prepared for Southern Blot analysis to confirm the single copy insertion as described supra (FIG. 3C).

The ES cell line with single copy cassette containing promoter-section-reporter is treated identically as described supra for differentiation and selection approach.

1. A method for selecting a first eukaryotic cell from a mixture of cell-types, which comprises the first eukaryotic cell and a second eukaryotic cell, wherein the mixture of cell-types is derived from a progenitor cell; the method comprising the steps of (a) contacting the progenitor cell with a first polynucleotide and a second polynucleotide which comprises a polynucleotide that encodes puromycin-N-acetyl-transferase and which is operably linked to a regulatory element, wherein the first polynucleotide and the second polynucleotide enter the progenitor cell and integrate into the progenitor cell genome at a predetermined site; (b) applying a first selective pressure to the progenitor cell, wherein a cell having the first polynucleotide integrated into the predetermined single site can survive; (c) allowing the cell having the first polynucleotide integrated into the predetermined single site to proliferate to produce a clonal population; (d) applying a growth condition to the clonal population, wherein cells within the clonal population begin the differentiate into one or more specific cell types to produce the mixture of cell-types; and (e) applying puromycin in an amount sufficient to kill the second eukaryotic cell to the mixture of cell-types, wherein the first eukaryotic cell survives and the second eukaryotic cell does not survive.

2. The method according to claim 1, wherein the first eukaryotic cell is a neuron.

3. The method according to claim 2, wherein the second eukaryotic cell is selected from the group consisting of glial cell, astrocyte, and oligodendrocyte.

4. The method according to claim 1, wherein the progenitor cell is a cell that gives rise to nervous tissue cells.

5. The method according to claim 4, wherein the progenitor cell is a stem cell.

6. The method according to claim 5, wherein the stem cell is an adult stem cell.

7. The method according to claim 5, wherein the stem cell is selected from the group consisting of germinal ridge cell, embryonal carcinoma cell, embryonic stem cell and fetal stem cell.

8. The method according to claim 5, wherein the stem cell is an embryonic stem cell.

9. The method according to claim 5, wherein the stem cell is a murine stem cell.

10. (canceled)

11. (canceled)

12. (canceled)

13. (canceled)

14. (canceled)

15. The method according to claim 1 wherein the regulatory element is a tissue-specific promoter.

16. The method according to claim 15 wherein the tissue-specific promoter is any one of neuron-specific promoter, beta-islet cell-specific promoter, muscle-specific promoter, cardiomyocyte-specific promoter, bone homeostasis-specific promoter, leukocyte-specific promoter, vascular endothelial cell-specific promoter, hepatocyte-specific promoter and lung epithelial cell-specific promoter.

17. The method according to claim 16 wherein the tissue-specific promoter is a neuron-specific promoter.

18. The method according to claim 17 wherein the neuron-specific promoter is necdin promoter or L7 promoter.

19. The method according to claim 18 wherein the neuron-specific promoter is necdin promoter, consisting of a sequence as set forth in SEQ ID NO:3.

20. (canceled)

21. The method according to claim 14 wherein the second polynucleotide further comprises a polynucleotide that encodes a polypeptide that produces a visual read-out.

22. The method according to claim 21 wherein the polypeptide that produces a visual read-out is beta-galactosidase and the visual read-out is the formation of a blue color.

23. A method for selecting a neuron from a mixture of cells derived from differentiating embryonic stem cells, comprising the steps of (a) transforming an embryonic stem cell with a first polynucleotide that confers neomycin resistance and a second polynucleotide that comprises comprising a neuron-specific promoter operably linked to a polynucleotide that encodes puromycin-N-acetyl-transferase, (b) next applying G418 to the embryonic stem cell, (c) next allowing the embryonic stem cell to proliferate to produce a clonal population, (d) next allowing the embryonic stem cell to differentiate to form the mixture of cells, and (e) contacting the mixture of cells with puromycin, wherein a neuron in the mixture produces puromycin-N-acetyl-transferase and survives, and any cell that is not a neuron is killed by the puromycin, thereby producing a second mixture of cells consisting of neurons.

24. The method according to claim 23 wherein the neuron-specific promoter is a necdin promoter having a sequence as set forth in SEQ ID NO:3.

25. The method according to claim 1 wherein the first polynucleotide comprises a neomycin resistance gene, the predetermined site is a hypoxanthine phosphoribosyl transferase (“HPRT”) locus, and the first selective pressure is the addition of G418 to the progenitor cell.


SEQUENCE LISTING (3 sequences)

SEQ ID NO: 1 - DNA, 26 bp, mammalian
gatcattttccactagaatcttaacg

SEQ ID NO: 2 - DNA, 20 bp, mammalian
tctgatccgaaggcgcagac

SEQ ID NO: 3 - DNA, 956 bp, mammalian
gatcattttccactagaatcttaacggaagctttagatgaaaacaaagtgacctaatagaaatggagagcccaaatctatagttattaaaataattcaaaaccaacaaactgagcatccaatgatgaatgaatgcatatatatatatattttatatatccctcattttcatgtggggcctgggggatttaaattctggttctcatgcttgaactgcaaaagctcttaaccactgaaccaagtctcattttaattaaaattttaaactcccatcaattatgctatatgaatctactacttctagaacattcaaatctcacatggatttatctccagtgtctgtaactacactttacattcttccttgtttgatctgcattcagatatgtaagaatttatctctattaataatttctcaatattccaatcttaaacaactcatcatcatcatcatcatcataaggtacagctttccaaaagaaaaaagaaaaaaaagtcatttctgtttctcttattcttctgtagaaaaaccaaaatcaagaattaagtcattctccaggaccttcacatttaatttgattttgcccaaactcagtgtgtccgaacttaacttcaccatctcagctacatctttctccttcaacttgtttctttccctaccatcatctagttctgtgccatacaggagaccagggaaatcttttacataagcctagtggtacccttcccttaagacccccattggttgggctttgactcttcggctcctttcccagccctaccactcttctggcttcccaacacgcatgcgcaatatcgcgtcagccccgcccgcccgctgctgcggaaggcgcagtgctcagtaaagcgcacttcctctgctggtctccaccgagggagtgcccgctccaagagctccaagccgcatcggtcctgctctgatccgaaggcgcagac